(19) United States
(12) Patent Application Publication
(10) Pub. No.: US 2018/0280100 A1
(43) Pub. Date: Oct. 4, 2018

(54) INSTRUMENT CONTROLLER FOR ROBOTICALLY ASSISTED MINIMALLY INVASIVE SURGERY

(71) Applicant: KONINKLIJKE PHILIPS N.V., EINDHOVEN (NL)

(72) Inventors: Aryeh Leib REINSTEIN, BRONX, NY (US); Aleksandra POPOVIC, BOSTON, MA (US); David Paul NOONAN, NEW YORK, NY (US)

(21) Appl. No.: 15/764,079

(22) PCT Filed: Sep. 29, 2016
(86) PCT No.: PCT/EP2016/073161

§ 371 Date: Mar. 28, 2018

Related U.S. Application Data
(60) Provisional application No. 62/234,100, filed on Sep. 29, 2015.

(51) Int. Cl. A61B 34/00; A61B 1/313; A61B 34/32; G06F 3/0354

(52) U.S. Cl. CPC A61B 34/74; A61B 1/3132; A61B 34/25; A61B 2034/301; G06F 3/03547; A61B 2034/742; A61B 34/32

(57) ABSTRACT

A robot control system includes a controller device (120) mountable on a medical instrument and including a housing with a trackpad configured to interact with a printed circuit board therein to create electronic signals corresponding with movement of the trackpad. The electronic signals include a point signal and a click signal. An adapter is formed on the housing and is configured to detachably connect the controller device to the medical instrument. A display (118) is responsive to the point signal of the controller device to permit a cursor to be moved on the display to indicate a position to be imaged or moved to. A robot system (144) is configured to respond to the click signal to move one of a robot directly or an instrument held by the robot in accordance with the position to be imaged or moved to.

### BACKGROUND

### Technical Field

This disclosure relates to medical instruments and more particularly to a robot control device mounted on a medical instrument to provide additional freedom to a surgeon.

### Description of the Related Art

Minimally invasive surgery is performed using elongated instruments inserted into a patient's body through small ports. Visualization during these procedures may include the use of an endoscope. In robotic guided minimally invasive surgery, one or more of the instruments is held and controlled by a robotic device.

A robotic arm holding an endoscope needs to be controlled and manipulated by a member of a surgery team. Often, a different member of the team, other than the surgeon, needs to perform the robotic manipulation, as the surgeon frequently has both hands occupied holding instruments. This can dramatically impact the surgical workflow, as the surgeon needs to describe to another team member how he/she wants the robot to move, and the other team member needs to implement the instructions received. Aside from the time delay this causes, miscommunications can also result further slowing down the surgery.

### SUMMARY

In accordance with the present principles, a controller device includes a base configured to support a printed circuit board and a button support positioned over the printed circuit board. A trackpad is supported by the button support and is configured to interact with the printed circuit board to create electronic signals corresponding with movement of the trackpad. An adapter is configured to detachably connect to a medical instrument such that the controller device remotely controls operation of at least one other instrument using the trackpad when mounted on the medical instrument.

A robot control system includes a controller device mountable on a medical instrument and including a housing with a trackpad configured to interact with a printed circuit board therein to create electronic signals corresponding with movement of the trackpad. The electronic signals include a point signal and a click signal. An adapter is formed on the housing and is configured to detachably connect the controller device to the medical instrument. A robot system is configured to respond to the click signal generated to move one of a robot or an instrument held by the robot in accordance with the point signal.

Another robot control system includes a controller device mountable on a medical instrument and including a housing with a trackpad configured to interact with a printed circuit board therein to create electronic signals corresponding with movement of the trackpad. The electronic signals include a point signal and a click signal. An adapter is formed on the housing and is configured to detachably connect the controller device to the medical instrument. A display is responsive to the point signal of the controller device to permit a cursor to be moved on the display to indicate a position to be imaged or moved to. A robot system is configured to respond to the click signal to move one of a robot directly or an instrument held by the robot in accordance with the position to be imaged or moved to.

Yet another robot control system includes a handheld medical instrument and a controller device mountable on the medical instrument and including a housing with a trackpad configured to interact with a printed circuit board therein to create electronic signals corresponding with movement of the trackpad. The electronic signals include a point signal and a click signal. An adapter is formed on the housing and configured to detachably connect the controller device to the medical instrument. The controller device is configured to permit access to the trackpad by a hand of a user while employing the medical instrument. A display is responsive to the point signal of the controller device to permit a cursor to be moved on the display to indicate a position to be imaged or moved to by an imaging device providing viewing content on the display. A robot system is configured to respond to the click signal to move the imaging device held by the robot in accordance with the position to be imaged or moved to such that the robot system is controlled by the user with the hand employing the medical instrument.

A method for remote control of a robot includes connecting (502) a controller device on a medical instrument, the controller device including a housing with a trackpad configured to interact with a printed circuit board therein to create electronic signals corresponding with movement of the trackpad, the electronic signals including a point signal and a click signal, and an adapter formed on the housing and configured to detachably connect the controller device to the medical instrument; and positioning a robot system in response to the click signal to move one of a robot or an instrument held by the robot to a position in accordance with the point signal.

These and other objects, features and advantages of the present disclosure will become apparent from the following detailed description of illustrative embodiments thereof, which is to be read in connection with the accompanying drawings.

### BRIEF DESCRIPTION OF DRAWINGS

This disclosure will present in detail the following description of preferred embodiments with reference to the following figures wherein:

FIG. 1 is a block/flow diagram showing a controller system, which employs a controller device to control robot position in accordance with one embodiment;

FIG. 2 is a perspective view showing a forward-facing controller device attached to a medical instrument in accordance with one embodiment;

FIG. 3 is a back view showing a back surface of the controller device for attaching to a medical instrument in accordance with one embodiment;

FIG. 4 is a side perspective view showing an interface adapter on a side surface of the controller device in accordance with one embodiment;

FIG. 5 is an exploded perspective view showing internal portions of the controller device in accordance with one embodiment;

FIG. 6 is a perspective view showing a side-facing controller device attached to a medical instrument in accordance with one embodiment; and

FIG. 7 is a flow diagram showing a method for remote control of a robot in accordance with illustrative embodiments.

### DETAILED DESCRIPTION OF EMBODIMENTS

In accordance with the present principles, devices and methods are provided that permit a surgeon to retain personal control of a robotic arm while still maintaining full dexterity and full use of both hands. In one embodiment, the robotic arm may hold, e.g., an endoscope. A controller device is provided that attaches to an instrument (e.g., a laparoscopic instrument) that the surgeon holds. A trackpad or joystick is provided on the controller device to permit the surgeon to move a virtual pointer on an endoscopic video screen. When the pointer is highlighted on the screen, a button on the device can be pressed, and the robot moves towards the point. In one embodiment, the selected point moves towards the center of the endoscopic view.

It should be understood that the present invention will be described in terms of medical instruments; however, the teachings of the present invention are much broader and are applicable to any devices medical or otherwise. In some embodiments, the present principles are employed in performing procedures or analyzing complex biological or mechanical systems. In particular, the present principles are applicable to procedures in biological systems in all areas of the body such as the lungs, gastro-intestinal tract, excretory organs, blood vessels, etc. The present principles are applicable to devices for training users for procedures or for other applications. The elements depicted in the FIGS. may be implemented in various combinations of hardware and software and provide functions which may be combined in a single element or multiple elements.

The functions of the various elements shown in the FIGS. can be provided through the use of dedicated hardware as well as hardware capable of executing software in association with appropriate software. When provided by a processor, the functions can be provided by a single dedicated processor, by a single shared processor, or by a plurality of individual processors, some of which can be shared. Moreover, explicit use of the term “processor” or “controller” should not be construed to refer exclusively to hardware capable of executing software, and can implicitly include, without limitation, digital signal processor (“DSP”) hardware, read-only memory (“ROM”) for storing software, random access memory (“RAM”), non-volatile storage, etc.

Moreover, all statements herein reciting principles, aspects, and embodiments of the invention, as well as specific examples thereof, are intended to encompass both structural and functional equivalents thereof. Additionally, it is intended that such equivalents include both currently known equivalents as well as equivalents developed in the future (i.e., any elements developed that perform the same function, regardless of structure). Thus, for example, it will be appreciated by those skilled in the art that the block diagrams presented herein represent conceptual views of illustrative system components and/or circuitry embodying the principles of the invention. Similarly, it will be appreciated that any flow charts, flow diagrams and the like represent various processes which may be substantially represented in computer readable storage media and so executed by a computer or processor, whether or not such computer or processor is explicitly shown.

Furthermore, embodiments of the present invention can take the form of a computer program product accessible from a computer-usable or computer-readable storage medium providing program code for use by or in connection with a computer or any instruction execution system. For the purposes of this description, a computer-usable or computer readable storage medium can be any apparatus that may include, store, communicate, propagate, or transport the program for use by or in connection with the instruction execution system, apparatus, or device. The medium can be an electronic, magnetic, optical, electromagnetic, infrared, or semiconductor system (or apparatus or device) or a propagation medium. Examples of a computer-readable medium include a semiconductor or solid state memory, magnetic tape, a removable computer diskette, a random access memory (RAM), a read-only memory (ROM), a rigid magnetic disk and an optical disk. Current examples of optical disks include compact disk—read only memory (CD-ROM), compact disk—read/write (CD-R/W), Blu-Ray™ and DVD.

Reference in the specification to “one embodiment” or “an embodiment” of the present principles, as well as other variations thereof, means that a particular feature, structure, characteristic, and so forth described in connection with the embodiment is included in at least one embodiment of the present principles. Thus, the appearances of the phrase “in one embodiment” or “in an embodiment”, as well any other variations, appearing in various places throughout the specification are not necessarily all referring to the same embodiment.

It is to be appreciated that the use of any of the following “/”, “and/or”, and “at least one of”, for example, in the cases of “A/B”, “A and/or B” and “at least one of A and B”, is intended to encompass the selection of the first listed option (A) only, or the selection of the second listed option (B) only, or the selection of both options (A and B). As a further example, in the cases of “A, B, and/or C” and “at least one of A, B, and C”, such phrasing is intended to encompass the selection of the first listed option (A) only, or the selection of the second listed option (B) only, or the selection of the third listed option (C) only, or the selection of the first and the second listed options (A and B) only, or the selection of the first and third listed options (A and C) only, or the selection of the second and third listed options (B and C) only, or the selection of all three options (A and B and C). This may be extended, as readily apparent by one of ordinary skill in this and related arts, for as many items listed.

It will also be understood that when an element is referred to as being “on” or “over” another element, it can be directly on the other element or intervening elements may also be present. In contrast, when an element is referred to as being “directly on” or “directly over” another element, there are no intervening elements present. It will also be understood that when an element is referred to as being “connected” or “coupled” to another element, it can be directly connected or coupled to the other element or intervening elements may be present. In contrast, when an element is referred to as being “directly connected” or “directly coupled” to another element, there are no intervening elements present.

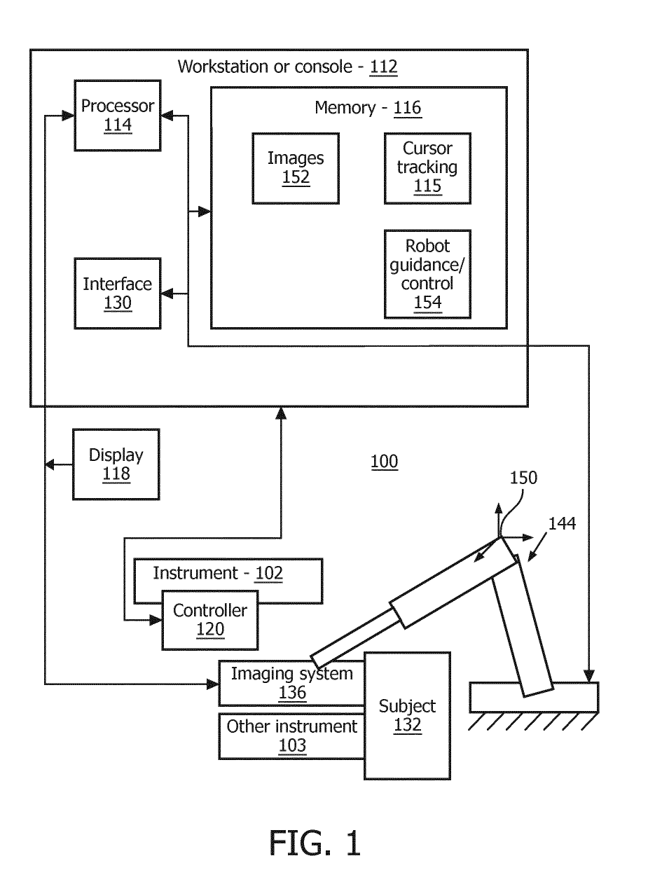

Referring now to the drawings in which like numerals represent the same or similar elements and initially to FIG. 1, a system 100 for controlling a robot using a controller attached to an instrument is illustratively shown in accordance with one embodiment. System 100 may include a workstation or console 112 from which a procedure is supervised and/or managed. Workstation 112 preferably includes one or more processors 114 and memory 116 for storing programs and applications.

A medical instrument 102 may include a laparoscopic instrument or any other instrument useful during a procedure, e.g., forceps, clamps, ablation electrodes, catheters, etc. The medical instrument 102 may be employed in conjunction with an imaging system 136. The imaging system or device 136 may include an endoscope or other scope configured to provide images 152 of an area of interest or an operable area. The medical instrument 102 includes a controller or controller device 120 mounted thereon. The controller 120 functions as a point and click device to reset a cursor position or image perspective on a display 118.

Memory 116 may store a cursor tracking program 115. The cursor tracking program 115 takes an input from the controller 120 to reset the cursor position on the display 118. In one embodiment, the cursor position can track the position of the controller 120 so that moving the controller 120 moves the cursor. In another embodiment, the cursor position is altered using the controller 120, and the imaging system 136 is moved in accordance with the cursor on the display 118. When a new cursor position is set using the controller 120, the cursor tracking program 115 identifies a new coordinate and relays this information to a robot 144 to correlate a position of the robot 144 holding the imaging device 136 (e.g., endoscope) with the cursor position on the display 118. The cursor position is preferably centrally maintained on the display 118 to facilitate workflow. The cursor position may be set to other locations (other than the center) on the display 118, as desired.

Position and orientation information about every point of the robot 144 relative to a reference location is known and can be provided, e.g., using encoders or other measurement systems. The position and orientation information of the robot 144 may be defined in terms of a robot coordinate system 150. The robot 144 may interact with a robot guidance/control system 154 tied into the workstation 112 to permit coordination between the controller 120 and the robot 144. A relationship between the robot 144 and the display 118 is correlated. Movement of a trackpad or other control on the controller 120 moves the cursor. Once the cursor is positioned properly, the controller 120 is activated (click). The cursor position is provided to cursor tracking 115, which translates the coordinates for the robot control system 154. The robot control system 154 send commands to control the robot 144 to reposition the imaging system 136 in accordance with the cursor position. One or more transforms may be employed to convey the cursor position to robot control commands.

Workstation 112 includes the display 118 for other purposes as well, e.g., viewing images (including preoperative images computed tomography (CT), magnetic resonance (MR), etc., real-time X-ray images, etc.) of a subject (patient) 132. The images may include images as overlays on other images (e.g., endoscope images on X-ray images, CT images, etc.). Display 118 may also permit a user to interact with the workstation 112 and its components and functions, or any other element within the system 100. This is further facilitated by an interface 130 which may include a keyboard, mouse, a joystick, a haptic device, or any other peripheral or control to permit user feedback from and interaction with the workstation 112.

In accordance with the present principles, a user or surgeon has personal control of the robot 144 holding the imaging device 136 (e.g., endoscope) so that exact actions can be performed as desired. In addition, the motion of the various joints in the robot 144 directly and intuitively relates to the motion that the surgeon truly desires, e.g., the motion of the endoscopic camera view. The surgeon can simply select a point on the screen of display 118 using the controller 120 to the position where the imaging point of view is desired thereby removing any complicated kinematics from the conscious workflow of the surgeon.

In another embodiment, the robot 144 is employed to hold another instrument 103 (e.g., a laparoscopic instrument) instead of the imaging device 136 endoscope. When the user selects a point on screen, instead of the robot 144 moving towards that point, the robot 144 moves the instrument 103 to that point. This would in effect give the surgeon a “third hand”, allowing him to manipulate three instruments at once.

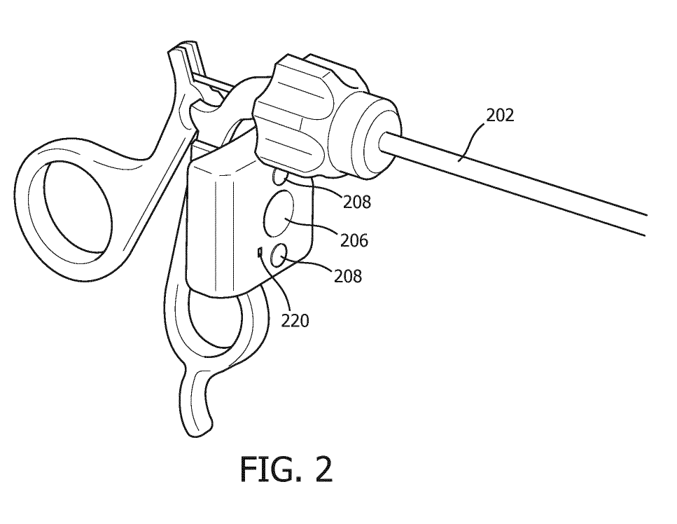

Referring to FIG. 2, an instrument 202, which permits a surgeon to retain personal control of a robotic arm that holds, e.g., an endoscope, while still maintaining full dexterity and full use of both hands, is illustratively shown. A controller device 220 (e.g., controller 120, in FIG. 1) mounts on the instrument 202, which may include a laparoscopic instrument in one embodiment. The surgeon holds the instrument 202 in one hand. A trackpad or joystick 206 is provided on the controller 220 to permit the surgeon to move a virtual pointer on an endoscopic video screen (e.g., display 118, FIG. 1).

In one embodiment, the instrument 202 includes a front or forward-facing controller device 220. The central trackpad 206 allows the user to move a pointer (cursor) on the endoscopic video feed and select a point to move towards by pressing down (clicking) on the central trackpad 206. Two buttons 208 provide additional functionality, such as display controls or the like. When the point that the surgeon desires is highlighted on the screen, a control mechanism (e.g., a click button on or below track pad 206) on the controller 220 is activated and the robot (144) moves towards the point, such that the selected point moves, e.g., towards the center of the endoscopic view.

The controller 220 includes trackpad 206 at a centrally disposed location. In one embodiment, the controller 220 faces along a length of the laparoscopic instrument (FIG. 2). This gives the controller an almost “trigger-like” feel, permitting the surgeon's index finger to wrap around the controller 220 and rest comfortably on the trackpad 206. The surgeon can still use other fingers normally to manipulate the laparoscopic instrument 202.

In another embodiment, the controller 220 can toggle between two modes. For example, a first mode may include selecting points on the endoscopic video feed and a second mode may provide for the direct manipulation of robotic joints. A button (e.g., button 208) can be pressed to toggle between the two modes. In another embodiment, a miniature “thumb” joystick may be substituted for the trackpad 206. A joystick may be easier to use if direct manipulation of the robotic joints is desired instead of selection of points on screen.

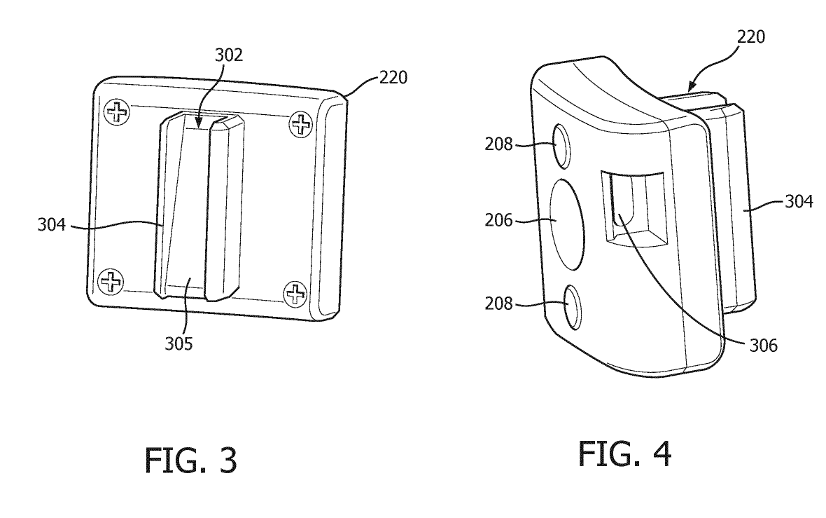

Referring to FIG. 3, a back view of the controller 220 is illustratively shown. The controller 220 includes an instrument interface 304. The instrument interface 304 may be removable so that it can be configured for different devices or instruments. In the embodiment shown, the interface 304 forms a channel 302 to provide a securing position to receive the instrument (e.g., a laparoscopic instrument). The channel 302 may include other materials or structures to assist in securing the instrument.

The controller 220 can have interchangeable adapters/interfaces 304 to permit the attachment to different instruments including, but not limited to laparoscopic instruments. In other embodiments, a magnetic adapter 305 may be employed to further secure the controller 220 to the instrument. In one embodiment, the magnetic adapter 305 may include a neodymium magnet that attaches to the metallic laparoscopic instrument handle. This adapter 305 permits for quick placement onto and off of the handle while still remaining firmly attached to the handle. In another embodiment, the controller can be manufactured as part of the laparoscopic handle itself.

Referring to FIG. 4, a side perspective view of the controller 220 shows illustrative functionality of the controller 220. The controller 220 may include an adapter interface 306 for connecting to a universal serial bus (USB) or other interface adapter port. The interface 306 may provide data from the trackpad 206 and/or buttons 208 to the workstation 112 to permit recording of commands, to transform the commands into robot motion or change of a display perspective as described above. In one embodiment, communication between the controller 220 and the workstation 112 is performed wirelessly. Each device may include an antenna or include a printed circuit board with an antenna and communicate using known communication protocols, e.g., BLUETOOTH™, etc.

The trackpad 206 can be employed to manipulate a virtual pointer or cursor on the endoscopic video feed (by wireless or wired signaling). When the desired point is selected, the user presses down on the trackpad 206, which serves as a button press, thereby initiating robotic motion. The robot then moves to center the selected point in the endoscopic video feed. If the user wants to stop the robot at any time, the user can press the trackpad button (206) at any time after motion has started. The additional buttons 208 can add other functionality, for example, additional degrees of motion such as, e.g., roll or zoom can be controlled using buttons 208 (e.g., one button can be used as zoom in, and the another as zoom out).

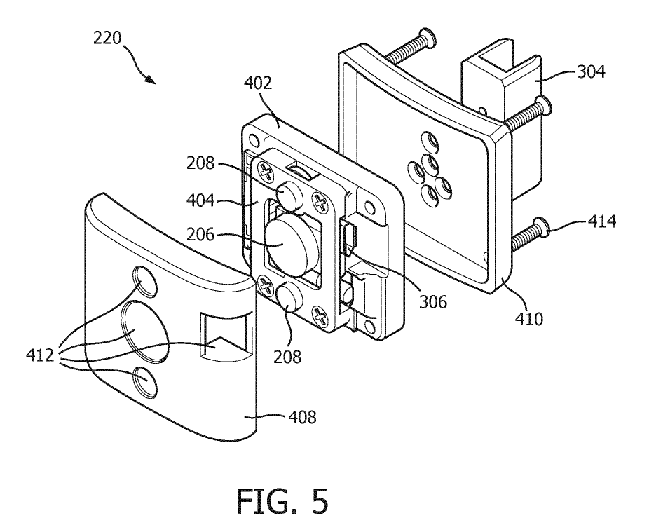

Referring to FIG. 5, an exploded perspective view of the controller 220 is shown. The exploded view shows a cover 408 removed to expose internal features of the controller 220. The cover 408 includes openings 412 to permit access to buttons 208, trackpad 206 and interface adapter 306. The controller 220 includes a base 402, which functions as a support for electronic components. The base 402 supports a printed circuit board (PCB) 404, and may be employed to store a battery or batteries for a wireless embodiment. A button support 406 is connected to the base 402 to provide support for buttons 208 and trackpad 206. The buttons 208 and trackpad 206 interface with the PCB 404 to create electronic signals corresponding to position commands from the trackpad 206, the depression of the trackpad as a button, and the depression of the buttons 208.

A back cover 410 engages the base 402 and is secured to the cover 408 by screws 414 or other mechanical devices. The interchangeable adapter/interface 304 is coupled to the back cover 410.

In particularly useful embodiments, the controller 220 communicates wirelessly with the controlling computer/workstation using BLUETOOTH™ (or other wireless protocol). This gives the user greater range of motion and overall freedom. Wireless communication employs a battery that is encased inside the controller 220, e.g., within the holder 402. If a wireless protocol is used, the controller 220 may provide charging of the battery through the interface adapter 306. In another embodiment, a powered wired connection is employed instead, reducing overall range of motion but insuring a constant reliable power source. Using a wired connection also reduces the overall size of the controller 220, as a battery is not needed. The controller 220 may include the capability for both wires and wireless operation.

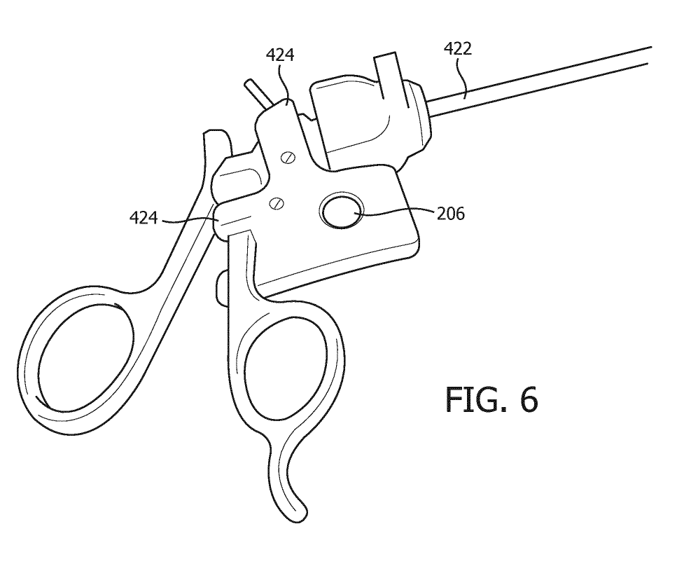

Referring to FIG. 6, in another embodiment, a controller 420 includes a sideways orientation, such that the trackpad 206 faces roughly perpendicular to a length of an instrument 422 (e.g., a laparoscopic instrument)). The embodiment shown is configured for a right handed user. While the front-facing controller is configured more universally for all users, the sideways orientation may be easier for some users to employ. A clip or clips 424 are employed to attach the controller 220 to the instrument 422. Other connection mechanisms including magnets are also contemplated.

The present principles are useful for giving a surgeon direct personal control of the robotic arm while freeing up his/her hands. Eye-tracking or oral dictation systems are not yet robust enough to guide the robot reliably, and foot pedals require additional dexterity and shifting of body weight in the midst of the surgery. While the present principles are particularly useful for laparoscopic surgery, the present principles may be employed in any number of procedures. Applications for robotic guided minimally invasive procedures may include cardiac surgery (e.g., atrial septal defect closure, valve repair/replacement, etc.); laparoscopic surgery (e.g., hysterectomy, prostactomy, etc.); gall bladder surgery; Natural Orifice Transluminal Surgery (NOTES); Single Incision Laparoscopic Surgery (SILS); pulmonary/bronchoscopic surgery; minimally invasive diagnostic interventions (e.g., arthroscopy); etc.

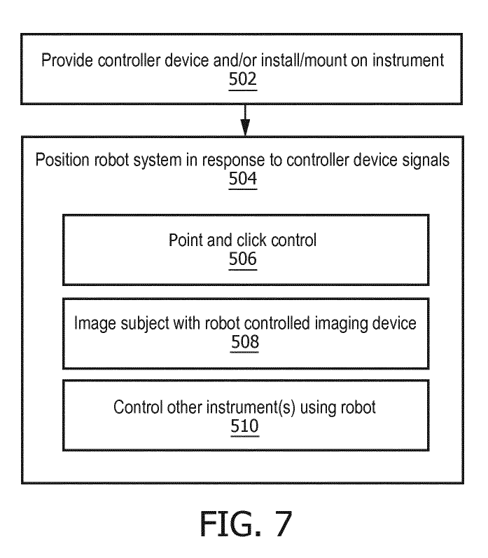

Referring to FIG. 7, a method for remote control of a robot is shown in accordance with illustrative embodiments. In block 502, a controller device is provided. The controller device includes a housing with a trackpad (or joystick) configured to interact with a printed circuit board to create electronic signals corresponding with movement of the track pad. The electronic signals include a point signal and a click signal. The controller device is configured to be mounted on a medical instrument and is installed or mounted on the medical instrument using an adapter that is configured to detachably connect the controller device to the medical instrument. The adapter may be configured to be detachably removed from the instrument using an attachment mechanism, such as, a magnet, clip, channel, or the like. In block 504, a robot system is positioned in response to the click signal to move one of a robot or an instrument held by the robot to a position in accordance with the point signal.

In block 506, the trackpad may include a point and click mechanism (e.g., trackpad and button). A cursor position may be controlled by pointing the cursor with the trackpad. The cursor position is provided on a display. By clicking the button of the trackpad, the cursor position is translated into a robot coordinate, and the robot and/or the instrument the robot is holding is moved to the corresponding cursor location.

In block 508, the instrument held by the robot may include an imaging device (e.g., an endoscope). A display image may be centered on the cursor (as the cursor is moved) by moving the robot with the imaging device with the movement of the cursor.

In block 510, the robot may be controlled using the controller, and/or an instrument held by the robot may be controlled using the controller device.

In interpreting the appended claims, it should be understood that:
a) the word “comprising” does not exclude the presence of other elements or acts than those listed in a given claim;
b) the word “a” or “an” preceding an element does not exclude the presence of a plurality of such elements;
c) any reference signs in the claims do not limit their scope;
d) several “means” may be represented by the same item or hardware or software implemented structure or function; and
e) no specific sequence of acts is intended to be required unless specifically indicated.

Having described preferred embodiments for instrument controller for robotically assisted minimally invasive surgery (which are intended to be illustrative and not limiting), it is noted that modifications and variations can be made by persons skilled in the art in light of the above teachings. It is therefore to be understood that changes may be made in the particular embodiments of the disclosure disclosed which are within the scope of the embodiments disclosed herein as outlined by the appended claims. Having thus described the details and particularity required by the patent laws, what is claimed and desired protected by Letters Patent is set forth in the appended claims.

1. A robot control system, comprising:
a controller device mountable on a medical instrument and including a housing with a trackpad configured to interact with a printed circuit board therein to create electronic signals corresponding with movement of the trackpad, the electronic signals including a point signal and a click signal, and an adapter formed on the housing and configured to detachably connect the controller device to the medical instrument;
a display responsive to the point signal of the controller device to permit a cursor to be moved on the display to indicate a position to be imaged or moved to; and
a robot system configured to respond to the click signal to move one of a robot directly or an instrument held by the robot in accordance with the position to be imaged or moved to.

2. The system as recited in claim 1, wherein the trackpad is centrally disposed on the controller device and faces distally along when attached to the medical instrument.

3. The system as recited in claim 1, wherein the trackpad is disposed sideways on the controller device and faces in a perpendicular direction to a longitudinal axis of the medical instrument, when attached to the medical instrument.

4. The system as recited in claim 1, wherein the trackpad includes a point and click mechanism such that the point mechanism moves the cursor on the display and the click mechanism activates the robot system to move one of the robot or the instrument held by the robot to the cursor location.

5. The system as recited in claim 1, wherein the instrument held by the robot includes an imaging device.

6. The system as recited in claim 1, wherein the adaptor includes one of a channel, a clip or a magnet to attach to the medical instrument.

7. The system as recited in claim 1, wherein the controller device communicates wirelessly with a workstation to move the cursor in accordance with the point signal and activate the robot system with the click signal.

8. The system as recited in claim 1, further comprising an interface adapter configured to provide a wired connection between the controller device and a workstation to move a cursor in accordance with the point signal and activate the robot system with the click signal.

9. The system as recited in claim 1, wherein the trackpad controls a cursor on a display and the cursor position is translated by a cursor tracking program to a position in a robot coordinate system.

10. A robot control system, comprising:
a handheld medical instrument;
a controller device mountable on the medical instrument and including a housing with a trackpad configured to interact with a printed circuit board therein to create electronic signals corresponding with movement of the trackpad, the electronic signals including a point signal and a click signal, and an adapter formed on the housing and configured to detachably connect the controller device to the medical instrument, the controller device being configured to permit access to the trackpad by a hand of a user while employing the medical instrument;
a display responsive to the point signal of the controller device to permit a cursor to be moved on the display to indicate a position to be imaged or moved to by an imaging device providing viewing content on the display; and
a robot system configured to respond to the click signal to move the imaging device held by the robot in accordance with the position to be imaged or moved to such that the robot system is controlled by the user with the hand employing the medical instrument.

11. The system as recited in claim 10, wherein the trackpad is centrally disposed on the controller device and faces distally along when attached to the medical instrument.

12. The system as recited in claim 10, wherein the trackpad is disposed sideways on the controller device and faces in a perpendicular direction to a longitudinal axis of the medical instrument, when attached to the medical instrument.

13. The system as recited in claim 10, wherein the trackpad includes a point and click mechanism such that the point mechanism moves the cursor on the display and the click mechanism activates the robot system to adjust in accordance with the cursor location.

14. The system as recited in claim 10, wherein the adaptor includes one of a channel, a clip or a magnet to attach to the medical instrument.

15. The system as recited in claim 10, wherein the controller device communicates wirelessly with a workstation to move the cursor in accordance with the point signal and activate the robot system with the click signal.

16. The system as recited in claim 10, further comprising an interface adapter configured to provide a wired connection between the controller device and a workstation to move a cursor in accordance with the point signal and activate the robot system with the click signal.

17. The system as recited in claim 10, wherein the trackpad controls a cursor on a display and the cursor position is translated by a cursor tracking program to a position in a robot coordinate system.

18. A method for remote control of a robot, comprising:
connecting a controller device on a medical instrument, the controller device including a housing with a trackpad configured to interact with a printed circuit board therein to create electronic signals corresponding with movement of the trackpad, the electronic signals including a point signal and a click signal, and an adapter formed on the housing and configured to detachably connect the controller device to the medical instrument; and
positioning a robot system in response to the click signal to move one of a robot or an instrument held by the robot to a position in accordance with the point signal.

19. The method as recited in claim 18, wherein the trackpad includes a point and click mechanism, the method further comprising pointing a cursor to a position on a display and clicking to activate the robot system to move one of the robot or the instrument held by the robot to the cursor location.

20. The method as recited in claim 19, wherein the instrument held by the robot includes an imaging device and the method further comprises centering a display image on the cursor by moving the robot with the imaging device.
